(19) United States
(12) Patent Application Publication
(10) Pub. No.: US 2024/0148314 A1
(43) Pub. Date: May 9, 2024

(54) PILLOW FOR PERFORMING ROLL MANEUVER

(71) Applicants: Michael Strupp, Graefelfing (DE); Christof Stocker, Graz (AT); Christoph Schöggler, Graz (AT); Anita Bhandari, Jaipur (IN); Rajneesh Bhandari, Jaipur (IN)

(72) Inventors: Michael Strupp, Graefelfing (DE); Christof Stocker, Graz (AT); Christoph Schöggler, Graz (AT); Anita Bhandari, Jaipur (IN); Rajneesh Bhandari, Jaipur (IN)

(21) Appl. No.: 18/370,100

(22) Filed: Sep. 19, 2023

(30) Foreign Application Priority Data
Nov. 3, 2022 (IN) 202241062967

(51) Int. Cl. A61B 5/00

(52) U.S. Cl. CPC A61B 5/4023; A61B 5/4884; A61B 5/706; A47G 9/10

(57) ABSTRACT

The present invention provides a pillow for performing roll maneuvers, wherein a subject performs the roll maneuver by following a set of instructions specified on the pillow. The pillow includes one or more segments that are sequentially arranged, wherein the pillow can be folded and unfolded at a central section along a vertical axis using a connecting element. Each segment of the one or more segments, with the set of instructions, is contoured on either side of a horizontal axis of the surface of the pillow. The set of instructions allows the subject to perform the roll maneuvers, which causes debris present in any position in the horizontal canal of the inner ear of the subject to move towards an utricle.

### TECHNICAL FIELD OF THE INVENTION

The present invention generally relates to a pillow for performing roll maneuvers. More particularly, the invention relates to the pillow for performing the roll maneuver, wherein a subject performs the roll maneuver by following a set of instructions specified on the pillow's surface for diagnosis and treatment of Horizontal Canal Benign Paroxysmal Positional Vertigo (HC-BPPV).

### BACKGROUND OF THE INVENTION

Benign Paroxysmal Positional Vertigo (BPPV), or positional vertigo, is one of the most common diseases of the inner ear that causes brief periods of vertigo triggered by changes in the position of the head, such as rolling, looking up, or bending down. BPPV is generally caused by small pieces of debris or crystals becoming displaced within the inner ear. Although not fatal in and of itself, the disease is often highly debilitating because the dizziness attacks appear random, can cause severe nausea, vomiting, and unfocused vision, and can interfere with normal daily activities such as driving and eating. BPPV is treated by moving a subject through a series of positions called a maneuver.

Several different maneuvers have been elaborated over decades for the treatment of HC-BPPV (Canalithiasis and Cupulolithiasis), which occurs when otoconia move within a semicircular canal, causing vertigo and nystagmus. Liberatory maneuvers include maneuvers such as the Roll maneuver, Gufoni maneuver, Forced prolonged position, and Zuma maneuvers. Canalithiasis, the free-floating otoconial debris, move due to gravitational pull, causing cupular deflection during head movement.

Conventional head maneuver exercises for BBPV patients generally have a set of attributes in common such as the duration of a session of the exercises lasts from several minutes to about 20 minutes, the exercises are carried out in discrete sessions even if the particular exercise is designed to be repeated periodically, the exercises are carried out when the patient has symptoms of BPPV, the exercises include orienting the patient's head in various positions, and having the head lying on a flat surface and having the head tilted back hanging over an edge of a bed.

Elevating the head while a person is resting or sleeping through the night has been recognized to provide potential wellness benefits to the resting or sleeping person. It is known that keeping the head elevated during a dizziness episode for patients diagnosed with BPPV and related diseases caused by conditions in the inner ear may help alleviate dizziness symptoms, and doctors sometimes recommend that patients sleep on a stack of pillows.

However, the actual head position beneficial to BPPV patients is not being elevated so much as being tilted with the top of the head at a higher position than the lower portion of the head. Moreover, some head maneuvering exercises repeatedly position the patient's head at certain angles from either horizontal or vertical positions for a short duration of under a minute at each position.

Currently, pillows are typically designed for a particular sleeping position, such as, for example, on the side, on the stomach, or on the back, based on the degrees of firmness of the pillow. Other types of pillows in the existing art that allow a subject or a patient to perform different types of head maneuvers mandate the presence and direct supervision of a trained professional or a corresponding healthcare advisor.

Moreover, even under the presence and direct supervision of the trained professional, there are high chances that the subject performing the head maneuvers is error-prone and inaccurate, leading to improper treatment of HC-BPPV Canalithiasis.

Accordingly, it is seen that a need exists for a pillow that can directly instruct a subject or a patient to perform the head/roll maneuver without any requirement of supervision and guidance of the trained professional, thereby avoiding inaccuracies and errors in sequence of steps of performing the roll maneuver and reducing efforts in the treatment of HC-BPPV.

Limitations and disadvantages of conventional approaches will become apparent to one of ordinary skill in the art through comparison of described systems with some aspects of the present invention, as outlined in the remainder of the present application and with reference to the drawings.

### SUMMARY OF THE INVENTION

According to an embodiment, a pillow is disclosed, which facilitates a subject or a patient to perform roll maneuver by following a set of instructions specified on the pillow as shown in and/or described in connection with, at least one of the figures. The embodiment may include specifying the set of instructions on the pillow for performing the roll maneuver for diagnosing and treating patients with Horizontal Canal Benign Paroxysmal Positional Vertigo (HC-BPPV).

The pillow includes a surface with one or more segments sequentially arranged on the surface. The one or more segments are contoured into various shapes to fit the head of the subject into respective positions during the roll maneuver of the subject while the pillow is in a flat position. Each segment of the one or more segments is contoured on either side of a horizontal axis of the pillow's surface. The one or more segments include a first segment, a second segment, a third segment, and a fourth segment. The second and fourth segments of the one or more segments are contoured to elevate a head position of a subject at various angles such as, for example, 25 degrees to cause the horizontal canal of the subject's inner ear to be vertical. The first segment is contoured to change the head position of the subject in specific positions to cause the horizontal canal of the inner ear of the subject to be vertical and thereby parallel to the gravitational vector. The set of instructions to perform the roll maneuver is specified on each segment of the one or more segments. The set of instructions includes markings that indicate ways to position the head of the subject on each respective segment, wherein the markings are on either side of the horizontal axis of the surface.

The set of instructions on the surface of the pillow allows the subject to perform the roll maneuvers at an angle range of 0 degrees to 360 degrees by sequentially positioning the head of the subject on the one or more segments. Performing the roll maneuver at an angle range of 0 degrees to 360 degrees on the pillow's surface causes debris in any position in the horizontal canal of the subject's inner ear to move towards an utricle of the inner ear.

Those and other features and advantages of the present invention may be appreciated from a review of the following detailed description of the present invention, along with the accompanying figures in which like reference numerals refer to like parts throughout.

### BRIEF DESCRIPTION OF THE FIGURES

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 provides a diagrammatic representation of a pillow in accordance with an embodiment of the present invention.

FIG. 2a provides an exemplary illustration of a subject performing the roll maneuver on the pillow on the first segment in accordance with an embodiment of the present invention.

FIG. 2b provides another exemplary illustration of the subject performing the roll maneuver on the pillow on the second segment in accordance with an embodiment of the present invention.

FIG. 2c provides yet another exemplary illustration of the subject performing the roll maneuver on the pillow on the third segment in accordance with an embodiment of the present invention.

FIG. 2d provides yet another exemplary illustration of the subject performing the roll maneuver on the pillow on the fourth segment in accordance with an embodiment of the present invention.

FIG. 3 illustrates a simulation of the movement of debris in the subject's inner ear while performing the roll maneuver on the pillow in accordance with an embodiment of the present invention.

FIG. 4 illustrates a flowchart of a method for performing the roll maneuver on the pillow in accordance with an embodiment of the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail embodiments that are in accordance with the present invention, it should be observed that the embodiments reside primarily in combinations of method steps and an apparatus related to a pillow for performing roll maneuver. Accordingly, the apparatus and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

Detailed embodiments of the claimed structures and methods are disclosed herein; however, it can be understood that the disclosed embodiments are merely illustrative of the claimed structures and methods that may be embodied in various forms. The invention may, however, be embodied in many different forms and should not be construed as limited to the exemplary embodiments set forth herein. In the description, details of well-known features and techniques may be omitted to avoid unnecessarily obscuring the presented embodiments.

It is to be understood that the singular forms “a”, “an”, and “the” include plural referents uncles the context clearly dictates otherwise. Thus, for example, reference to “a component surface” includes reference to one or more of such surfaces unless the context clearly dictates otherwise. For purposes of description herein, the terms “upper”, “lower”, “left”, “rear”, “right”, “front”, “vertical”, “horizontal”, and derivatives thereof shall relate to the present invention.

In this document, relational terms, if any, such as first and second, top and bottom, and the like may be used solely to distinguish one entity or action from another entity or action without necessarily requiring or implying any relationship or order between such entities or actions. The terms “comprises,” “comprising,” or any other variation thereof are intended to cover a nonexclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

Generally speaking, pursuant to various embodiments, the invention is related to the pillow, and more particularly related to the pillow for performing the roll maneuver. The following described exemplary embodiments provide an apparatus, and method to perform the roll maneuver by following the set of instructions specified on a surface of the pillow. Therefore, the present embodiment can improve the diagnosis and treatment of Horizontal Canal Benign Paroxysmal Positional Vertigo (HC-BPPV) by ensuring a subject performs the roll maneuver based on the set of instructions specified on the surface of the pillow.

The roll maneuver performed by the subject on the pillow utilizes gravitational force to move debris in the subject's inner ear through different positions out of the canal. The pillow of the present invention facilitates the subject to perform the roll maneuver in a sequence of steps which makes the semi-circular canal from horizontal to vertical position at each step, thereby moving the particle through the semi-circular canal due to gravity.

The following described implementations may be found in the disclosed pillow to perform the roll maneuver. A set of instructions specified on the pillow's surface facilitates the subject to perform the roll maneuver for diagnosing and treating patients with HC-BPPV.

The pillow comprises one or more segments that are sequentially arranged on the surface of the pillow. The one or more segments are contoured into different shapes to fit the head of the subject into respective positions during the roll maneuver of the subject while the pillow is in a flat position. Each segment of the one or more segments is contoured on either side of a horizontal axis of the surface of the pillow. The one or more segments include a first segment, a second segment, a third segment, and a fourth segment. The second and fourth segments of the one or more segments are contoured to elevate a head position of a subject at various angles such as, for example, 25 degrees to cause the horizontal canal of the subject's inner ear to be vertical. The first segment is contoured to change the head position of the subject in certain positions to cause the horizontal canal of the inner ear of the subject to be vertical and thereby parallel to the gravitational vector. The set of instructions to perform the roll maneuver is specified on each segment of the one or more segments. The set of instructions includes markings that indicate ways to position the head of the subject on each respective segment, wherein the markings are on either side of the horizontal axis of the surface.

The set of instructions on the surface of the pillow allows the subject to perform the roll maneuvers at an angle range of 0 degrees to 360 degrees by sequentially positioning the head of the subject on the one or more segments. Performing the roll maneuver at an angle range of 0 degrees to 360 degrees on the pillow's surface causes debris in any position in the horizontal canal of the subject's inner ear to move towards an utricle of the inner ear.

In an embodiment, the pillow is configured to be folded into a compact shape, making it easy to store or carry around.

The pillow is foldable at a central section along a vertical axis at an angle of 90 degrees. The one or more segments of the pillow overlap and face one another while the pillow is in the folded configuration. For instance, the first segment and the fourth segment of the pillow overlap with one another, and the second segment and the third segment overlap with one another in the folded configuration. When the pillow is unfolded, the one or more segments of the pillow lie in a flat position. The first segment, the second segment, the third segment, and the fourth segment are engaged sequentially in the flat position while the pillow is in the unfolded configuration.

In another embodiment, the pillow is made up of individual segments such as the first segment, the second segment, the third segment, and the fourth segment. These individual segments are engaged with one another using a snap-fit mechanism to make a surface of the pillow. In an exemplary implementation, the first and second segments are engaged using the snap-fit mechanism to form one module, and the third and fourth segments are similarly engaged to form another module. Thereafter, these modules are engaged with each other to form the surface of the pillow. In an embodiment, such an entire assembly of these modules may be configured to be foldable at the central section along the vertical axis at an angle of 90 degrees.

As known in the art, the snap-fit mechanism is an arrangement method used to attach flexible parts, usually plastic, to form the final product by pushing the parts' interlocking components together. There are several variations in snap-fits, including cantilever, torsional and annular. Snap-fits, as integral attachment features, are an alternative to assembly using nails or screws and have the advantages of speed and no loose parts.

Various aspects of the present invention are described by narrative text, flowchart, illustrations of the pillow. With respect to any flowcharts, depending upon the mechanism involved, the operations can be performed in a different and/or other order than what is shown in a given flowchart.

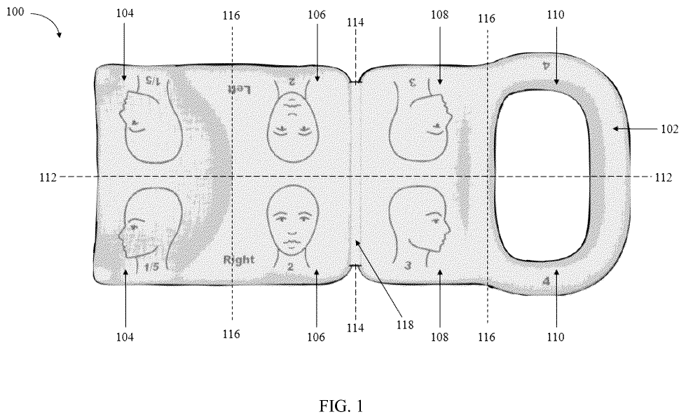

FIG. 1 provides a diagrammatic representation of a pillow 100 in accordance with an embodiment of the present invention. Referring to FIG. 1, the pillow 100 includes a surface 102, a first segment 104, a second segment 106, a third segment 108, a fourth segment 110, a horizontal axis 112, a vertical axis 114, one or more snap-fit hinges 116, and a connecting element 118.

The surface 102 of the pillow 100 engages the first segment 104, the second segment 106, the third segment 108, and the fourth segment 110 along the horizontal axis 112.

The segments 104-110 are contoured to have different shapes to fit a subject's head into respective positions during the roll maneuver, while the pillow 100 is in the flat position. The first segment 104, the second segment 106, the third segment 108, and the fourth segment 110 are contoured on either side of the horizontal axis 112 of the surface 102 of the pillow 100. Contouring the segments 104-110 on either side of the horizontal axis 112 facilitates the use of the pillow 100 to perform the roll maneuver on either side of the pillow, depending on whether the left or right ear needs to be treated.

In an exemplary embodiment, the first segment 104, the second segment 106, the third segment 108, and the fourth segment 110 of the pillow 100 are made of various types of materials such as, but not limited to, a foam material, a wool bolus material, a microfiber material, a down cluster, and a latex material. The materials mentioned above provide a range of density from soft, medium, and firm. Alternatively, the materials can be such as, but not limited to, cotton, feathers, down, polyester, the combination of feather and down, husks, beads, beans, pits, foam variants (e.g., molded foam material, shaped foam material, or other suitable yielding material) or any combination or any other known material.

The first segment 104 on the surface 102 of the pillow 100 is contoured into a shape that fits the head of the subject and allows changing the head position of the subject in certain positions to cause the membranous labyrinth of the horizontal canal of the inner ear of the subject to be vertical and thereby parallel to a gravitational vector. The gravitational vector is a vector field that describes the gravitational force that would be applied to an object or a subject at any given point in space per unit of mass.

The second segment 106 on the surface 102 of the pillow 100 is contoured to elevate the head position of the subject to 25 degrees from the gravitational vector to cause the horizontal canal of the subject's inner ear to be vertical.

In some non-limiting embodiments, the second segment 106 on the surface 102 of the pillow 100 is contoured to elevate the head position of the subject at various other angles to cause the horizontal canal of the subject's inner ear to be vertical.

The third segment 108 on the surface 102 of the pillow 100 is contoured to change the head position of the subject in certain positions to cause the membranous labyrinth of the horizontal canal of the subject's inner ear to be vertical, thereby parallel to the gravitational vector.

The fourth segment 110 on the surface 102 of the pillow 100 is contoured to elevate the head position of the subject to 25 degrees from the gravitational vector to cause the horizontal canal of the subject's inner ear to be vertical.

In some non-limiting embodiments, the fourth segment 110 on the surface 102 of the pillow 100 is contoured to elevate the head position of the subject at various other angles to cause the horizontal canal of the subject's inner ear to be vertical.

In an embodiment, the pillow 100 is foldable at the central section along the vertical axis 114 using the connecting element 118. The connecting element 118 can be such as, but not limited to, foldable hinges, foldable frames, and foldable hooks.

The pillow 100 is foldable at the central section along the vertical axis 114 at 90 degrees. The one or more segments of the pillow 100 overlap and face one another while the pillow 100 is in the folded configuration. For instance, in a folded configuration, the first segment 104 and the fourth segment 110 of the pillow 100 overlap with one another, and the second segment 106 and the third segment 108 overlap. When the pillow 100 is unfolded, the one or more segments of the pillow 100 lie in a flat position. The first segment 104, the second segment 106, the third segment 108, and the fourth segment 110 are engaged sequentially in the flat position, while the pillow 100 is in the unfolded configuration.

In another embodiment, the pillow 100 is made up of the individual segments such as the first segment 104, the second segment 106, the third segment 108, and the fourth segment 108. These individual segments are engaged with one another using a snap-fit mechanism to make a surface of the pillow. In an exemplary implementation, the first segment 104 and the second segment 106 are engaged using the snap-fit mechanism to form one module, and the third segment 108 and fourth segment 110 are similarly engaged in forming another module. Thereafter, these modules are engaged with each other to form the surface of the pillow 100. In an embodiment, such an entire assembly of these modules may be configured to be foldable at the central section along the vertical axis 114 at 90 degrees.

The first segment 104, the second segment 106, the third segment 108, and the fourth segment 110 enable a subject to perform the roll maneuver on the surface 102 of the pillow 100 at an angle range of 0 degrees to 360 degrees by sequentially positioning and moving the head of the subject. Performing the roll maneuvers at an angle range of 0 degrees to 360 degrees on the surface 102 of the pillow 100 causes debris present in any position in the horizontal canal of the subject's inner ear to move towards an utricle of the inner ear of the subject.

In an exemplary embodiment, the pillow 100 may be fashioned from a washable fabric material that may be selected from a plurality of fabrics such as, but are not limited to, cotton, rayon, silk, ballistic nylon, polyester, or blends thereof.

The pillow 100 may have a generally rectangular shape of the pillow as known in the art, and the dimensions of the pillow 100 measure in accordance with the pillows known in the art such as, but not limited to, a standard pillow, a queen size pillow, a king size pillow, and a body size pillow.

In another exemplary embodiment, the pillow 100 comprises various dimensions measurements in its flat position. The dimensions are exemplary and do not limit the size of the pillow 100 in any respect. Accordingly, the size of the pillow 100 may be altered and each of the dimensions may vary plus or minus a range of measurements, including, but not limited to, a thickness of 75 mm, a width of 300 mm, and a length of 600 mm.

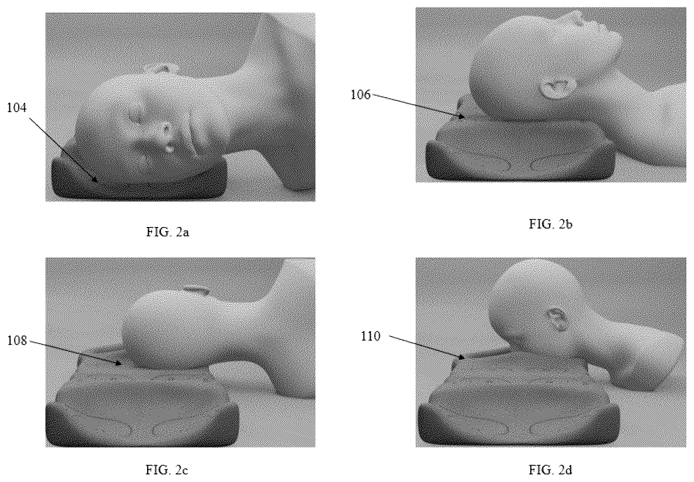

FIG. 2a provides an exemplary illustration of a subject performing the roll maneuver on the pillow 100 on the first segment 104 in accordance with an embodiment of the present invention. Following the set of instructions specified on the surface 102 of the pillow 100, the subject positions the head on the first segment 104.

The first segment 104 is contoured into a shape that allows the subject to place the head in a certain position to cause the membranous labyrinth of the horizontal canal of the subject's inner ear to be vertical and thereby parallel to the gravitational vector.

FIG. 2b provides an exemplary illustration of the subject performing the roll maneuver on the pillow 100 on the second segment 106 in accordance with an embodiment of the present invention. Following the set of instructions specified on the surface 102 of the pillow, the subject positions the head on the second segment 106.

The second segment 106 is contoured into a shape that allows the subject to place the head by elevating the head position to 25 degrees from the gravitational vector to cause the horizontal canal of the subject's inner ear to be vertical.

In some non-limiting embodiments, the second segment 106 is contoured into a shape that allows the subject to place the head by elevating the head position at various other angles to cause the horizontal canal of the subject's inner ear to be vertical.

FIG. 2c provides an exemplary illustration of the subject performing the roll maneuver on the pillow 100 on the third segment 108 in accordance with an embodiment of the present invention. Following the set of instructions specified on the surface 102 of the pillow, the subject positions the head on the third segment 108.

The third segment 108 is contoured into a shape that allows the subject to place the head at an angle of zero degrees from the gravitational vector to cause the horizontal canal of the subject's inner ear to be vertical.

FIG. 2d provides an exemplary illustration of the subject performing the roll maneuver on the pillow 100 on the fourth segment 110 in accordance with an embodiment of the present invention. Following the set of instructions specified on the surface 102 of the pillow, the subject positions the head on the fourth segment 110.

The fourth segment 110 is contoured into a shape that allows the subject to place the head by elevating the head position to 25 degrees from the gravitational vector to cause the horizontal canal of the subject's inner ear to be vertical.

In some non-limiting embodiments, the fourth segment 110 is contoured into a shape that allows the subject to place the head by elevating the head position at various other angles to cause the horizontal canal of the subject's inner ear to be vertical.

As the subject completes the roll maneuver at the fourth segment 110 of the pillow 100, the gravitational force moves the debris in the inner ear of the subject through the canal and brings it in the direction of the utricle of the inner ear.

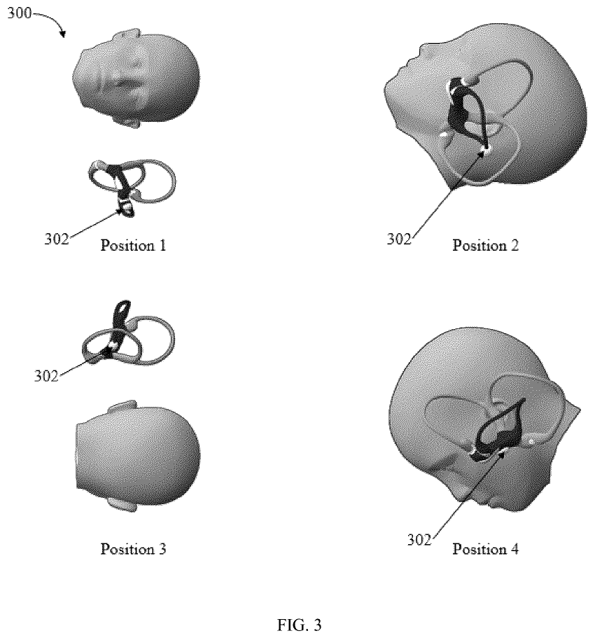

FIG. 3 illustrates a simulation 300 of the movement of debris 302 from position 1 to position 4 in the subject's inner ear while performing the roll maneuver on the pillow 100 per an embodiment of the present invention.

In certain non-limiting embodiments, the simulation 300 is a 3D model of the inner ear based on reconstructed MRI images of the temporal bone, wherein the 3D image of the inner ear is extracted from DICOM files of MRI images. Referring to FIG. 3, the debris 302 moves through the canal of the inner ear of the subject while the head of the subject is moved to different positions, namely position 1 (the first segment 102), position 2 (the second segment 104), position 3 (the third segment 106), and position 4 (the fourth segment 106).

While the subject performs the roll maneuver and moves the head from position 1 to position 4, based on the set of instructions specified on the surface of the pillow 100, the gravitational force moves the debris 302 in the inner ear of the subject through the canal and brings it in the direction of utricle of the inner ear.

In an embodiment, the roll maneuver for HC-BPPV-ca starts with a 270 degrees roll of the head and 90 degrees roll of the subject's body, starting with the subject in the supine position. As known in the art, in the supine position, the subject is faced up with the head resting on a pad positioner and the neck in a neutral position. The subject's arms, maintained in a neutral thumb-up or supinated position, may be tucked at the sides or abducted to less than 90 degrees on arm boards.

As illustrated in FIG. 3, the head of the subject is raised by 30 degrees to make the horizontal canal vertical. The roll maneuver uses gravity to move the debris 302 through the canal and brings it toward the utricle. Subsequently, the subject turns 90 degrees to the healthy side, moves into the prone position, and then turns another 90 degrees to the affected side. As known in the art, the subject lies flat on their stomach in the prone position. In other words, the prone position is where the subject is positioned face-down with the head in a neutral position without excessive flexion, extension, or rotation.

In an embodiment, the roll maneuver disclosed in the present invention works when the debris 302 is present in the non-ampullary arm of the horizontal canal. As illustrated in FIG. 3, the simulation demonstrates the movement of the debris 302 present in the non-ampullary arm of the horizontal canal by performing the roll maneuver. The debris 302 present in this position would cause geotropic nystagmus on the supine roll test, wherein geotropic nystagmus refers to nystagmus beating toward the ground. As mentioned above, when the subject's head is turned toward the affected side, the cupula will undergo an inhibitory deflection causing apogeotropic nystagmus.

In another embodiment, by modifying the positions of the subject, the roll maneuver disclosed in the present invention can provide treatment to the subject when the debris 302 is present in both ampullary and non-ampullary arms of the horizontal canal. The modified positions of the subject may include turning the head to the affected side first, returning the head to the central supine position, and following up with the steps described in the roll maneuver to enable the debris 302 present in any position in the horizontal canal to move towards the utricle.

The simulation, thereafter, shows the movement of the debris 302 in each step of HC-BPPV when the debris 302 is present in the ampullary arm close to the cupula in which apogeotropic nystagmus occurs. It is observed from the simulation that by turning the head of the subject to the affected side and performing the roll maneuver as a 360° rotation, the roll maneuver would be effective in treating BPPV with debris 302 in any position in the horizontal canal.

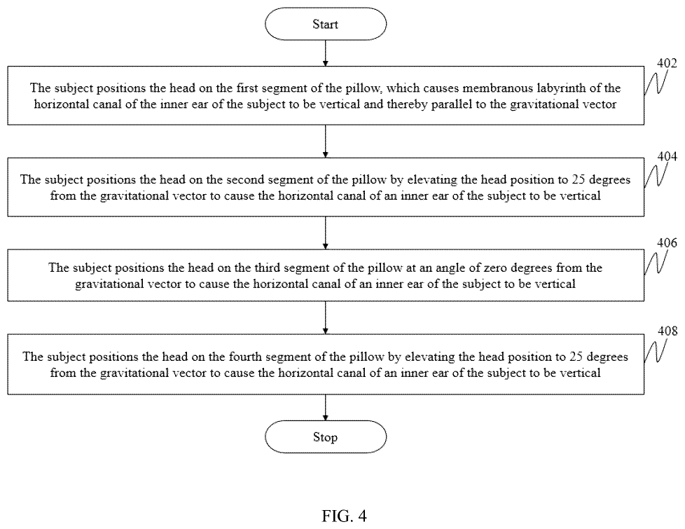

FIG. 4 illustrates a flowchart of a method for performing roll maneuvers on the pillow 100 in accordance with an embodiment of the present invention.

At step 402, the subject positions the head on the first segment 104 of the pillow 100, which causes the membranous labyrinth of the horizontal canal of the subject's inner ear to be vertical and thereby parallel to the gravitational vector.

The gravitational vector is a vector field that describes the gravitational force that would be applied to an object or a subject at any given point in space per unit of mass.

At step 404, the subject positions the head on the second segment 106 of the pillow 100 by elevating the head position to 25 degrees from the gravitational vector to cause the horizontal canal of the subject's inner ear to be vertical.

In some non-limiting embodiment, the subject positions the head on the second segment 106 of the pillow 100 by elevating the head position at various other angles to cause the horizontal canal of the subject's inner ear to be vertical.

Further, at step 406, the subject positions the head on the third segment 108 of the pillow 100 at an angle of zero degrees from the gravitational vector to cause the horizontal canal of the subject's inner ear to be vertical.

Finally, at step 408, the subject positions the head on the fourth segment 110 of the pillow 100 by elevating the head position to 25 degrees from the gravitational vector to cause the horizontal canal of the subject's inner ear to be vertical.

In some non-limiting embodiments, the subject positions the head on the fourth segment 110 of the pillow 100 by elevating the head position at various other angles to cause the horizontal canal of the subject's inner eat to be vertical.

The steps described in the roll maneuver enable the debris 302 present in any position in the horizontal canal of the inner ear to move towards the utricle.

Advantageously, the pillow disclosed in the present invention with uniquely contoured shapes facilitate patients or subjects to perform roll maneuver directly without needing any trained medical professional or a healthcare advisor. The patients using the pillow can efficiently perform the roll maneuver by reading the instructions specified on the pillow, which avoids the requirement of the presence of any trained medical professional on the site.

Another advantage of the present invention is that the pillow not only provides treatment to patients suffering from Horizontal Canal Benign Paroxysmal Positional Vertigo but also for early diagnosis of vertigo.

Yet another advantage of the present invention is that the pillow facilitates the treatment of Horizontal Canal Benign Paroxysmal Positional Vertigo of both ampullary and non-ampullary arms along with HC-cupulolithiasis. By sequentially providing instructions to the patients to turn the head to the affected side first and perform the roll maneuver at 360-degree rotation, the pillow ensures that the roll maneuver would effectively treat BPPV with debris in any position in the horizontal canal.

Yet another advantage of the present invention is that the pillow is foldable and compact, making it easy for a user to store or carry it around.

Those skilled in the art will realize that the above-recognized advantages and other advantages described herein are merely exemplary and are not meant to be a complete rendering of all of the advantages of the various embodiments of the present invention.

In the foregoing complete specification, specific embodiments of the present invention have been described. However, one of ordinary skill in the art appreciates that various modifications and changes can be made without departing from the scope of the present invention. Accordingly, the specification and figures are to be regarded in an illustrative rather than a restrictive sense, and all such modifications are intended to be included within the scope of the present invention.

1. A pillow, comprising:
a surface comprising a plurality of segments that are sequentially arranged on the surface, wherein the plurality of segments are contoured to bring the head of a subject into respective positions during a roll maneuver of the subject while the pillow is in a flat position.

2. The pillow as claimed in claim 1, wherein each segment of the plurality of segments is contoured on either side of a horizontal axis of the surface of the pillow.

3. The pillow as claimed in claim 1, wherein the plurality of segments are made of a range of materials comprising at least one of a foam material, a wool bolus material, a microfiber material, a down cluster, and a latex material.

4. The pillow as claimed in claim 1, wherein the pillow, in the flat position, comprises a plurality of dimensions such as, but not limited to, a thickness of 75 mm, a width of 300 mm, and a length of 600 mm.

5. The pillow as claimed in claim 1, wherein the plurality of segments comprise a first segment, a second segment, a third segment, and a fourth segment.

6. The pillow as claimed in claim 5, wherein the second segment and the fourth segment are contoured to elevate a head position of a subject at a plurality of angles such as, but not limited to, 25 degrees from the gravitational vector to cause the horizontal canal of an inner ear of the subject to be vertical.

7. The pillow as claimed in claim 6, wherein the first segment is contoured to change the head position of the subject in certain positions to cause the membranous labyrinth of the horizontal canal of the subject's inner ear to be vertical and thereby parallel to the gravitational vector.

8. The pillow as claimed in claim 1, wherein a set of instructions to perform the roll maneuver is specified on each segment of the plurality of segments, wherein the set of instructions comprises a marking to position the head of the subject on a corresponding segment, wherein the marking is on either side of the horizontal axis of the surface.

9. The pillow as claimed in claim 8, wherein the set of instructions are for performing the roll maneuver for diagnosing and treating patients with horizontal canal Benign paroxysmal positional vertigo (hcBPPV).

10. The pillow as claimed in claim 1, wherein each segment of the plurality of segments is adapted to be engaged with one or more other segments of the plurality of segments to form the surface of the pillow.

11. The pillow as claimed in claim 1, wherein each segment of the plurality of segments is adapted to be engaged with one or more other segments of the plurality of segments to form the surface of the pillow.

12. The pillow as claimed in claim 11, wherein each segment of the plurality of segments is adapted to be engaged with one or more other segments of the plurality of segments using a snap-fit mechanism.

13. A method for using a pillow configured for a roll maneuver, comprising:
performing the roll maneuver on a surface of the configured pillow at an angle range of 0 degrees to 360 degrees;
sequentially positioning a head of a subject on a plurality of segments of the configured pillow; and
rolling the head on the plurality of segments to move debris present in a horizontal canal of an inner ear of the subject towards an utricle of the inner ear of the subject.

14. The method for using the pillow as claimed in claim 13, wherein performing the roll maneuver comprises maintaining the orientation of the horizontal canal of the inner ear of the subject parallel to the earth vector during each of multiple head positions.
